(19) United States
(12) Patent Application Publication
(10) Pub. No.: US 2001/0005772 A1
(43) Pub. Date: Jun. 28, 2001

(54) Home doctor system, blood capsule and injection appliance

(75) Inventor: Toshiro Kisakibaru, Tokyo (JP)

Correspondence Address: RADER FISHMAN & GRAUER PLLC, LION BUILDING, 1233 20TH STREET N.W., SUITE 501, WASHINGTON, DC, 20036, US

(73) Assignee: Sony Corporation

(21) Appl. No.: 09/740,792

(22) Filed: Dec. 21, 2000

(30) Foreign Application Priority Data
Dec. 28, 1999 (JP) P11-374230
Sep. 27, 2000 (JP) P2000-293715

(51) Int. Cl. A61B 5/00

(52) U.S. Cl. 600/309; 600/576

(57) ABSTRACT

A subscriber of a home doctor center carries a physical condition-monitoring device. Physical condition data measured by the device is transmitted to a personal computer in the subscriber's residence, and then transmitted therefrom to the center via a contact path. Blood collected to the subscriber is sent to the center. The center analyzes the physical condition data and tests the received blood with an automatic test system. Based on analytic results of the physical condition data and results of blood tests, initial diagnosis of the subscriber is performed, and the subscriber is informed of the result of the initial diagnosis together with a corresponding advise. By close tie-up with a medial institute and an insurance institute, the center can early find diseases of subscribers, and by calculating premiums imposed to subscribers with reference to results of initial diagnosis, premiums can be reduced.

### BACKGROUND OF THE INVENTION

1. Field of the Invention

This invention relates to a home doctor system, blood capsule and injection appliance.

2. Description of the Related Art

People's interests in healthcare are getting larger and larger. As a result, market of dietary facilities, healthcare food and healthcare appliances has recently expanded rapidly. In regard to dietary conjunction, there are tonometers, pedometers, body fat measuring devices, and so forth, available for private use. Regarding healthcare food, a huge variety of preparations are commercially available.

However, these dietary facilities, healthcare food and healthcare appliances often have only weak medical support about their efficacies, and consumers cannot get valuable effects for expenses. Additionally, most of dietary facilities, healthcare food and healthcare appliances were sold as products, individually, and were far from the concept of systematic, continuous healthcare. On the other hand, along with developments of genetic engineering, some genetic diseases are now being specified by DNA decoding, but such data is not yet used for systematic healthcare.

Furthermore, currently available medical systems, medical institutions, medical care expenditure and insurance systems do not always meet the demand of people particularly interest in healthcare, and they involve problems, such as being directed at symptomatic therapy, requiring high expenditure for medical services, and so on. Additionally, inmost of insurance (life insurance, accident insurance, medial insurance) systems, subscribing conditions and insurance premiums are usually determined depending upon ages at the time of subscription and health information. However, considering that the state of health changes day to day, such calculation systems of insurance premiums are not rational.

On the other hand, data of blood test is relatively important for grasping the state of health. Blood collection heretofore relied on injectors. Injectors, however, are hypodermic injectors for collecting blood from veins, and normally cause tolerable pains. Causes of pains lie in needles being stuck deep, movements of physician or nurse's hands being transmitted to parts of bodies where needles stuck into, or other like reasons. There was also the problem that each injection or blood collection was conducted with a single needle and therefore takes a time.

Furthermore, blood was heretofore collected each time by an amount considerably larger than the necessary amount. Additionally, upon actual blood test, it was necessary to divide the collected blood for individual test items, and blood test could not conducted speedily.

### OBJECTS AND SUMMARY OF THE INVENTION

It is therefore an object of the invention to provide a home doctor system capable of removing current problems concerning healthcare and medical systems, medical institutions, medical expenditures and insurance systems.

A further object of the invention is to provide a blood capsule not requiring collection of an excessive amount of blood and capable of improving the blood collecting speed.

A still further object of the invention is to provide an injection appliance capable of reducing pains and other loads upon injection and blood collection.

According to the first aspect of the invention, there is provided a home doctor system characterized in that: data can be transmitted between a home doctor center and a plurality of subscribers, and each subscriber measures his or her physical condition data with a physical monitoring device and send it to the home doctor center; and the home doctor center makes initial diagnosis of the subscriber's physical condition from the physical condition data, and inform the subscriber of the result of the initial diagnosis.

According to the second aspect of the invention, there is provided a home doctor system characterized in that: data can be transmitted between a home doctor center and a plurality of subscribers, and each subscriber send his or her blood to the home doctor center; and the home doctor center executes blood tests of the blood, makes initial diagnosis of the subscriber from results of the blood tests, and inform the subscriber of a result of the initial diagnosis.

According to the third aspect of the invention, there is provided a home doctor system comprising a home doctor center, a blood test center and a blood-collecting site that are connected together, the blood-collecting site sending blood to the blood test center; and results of tests executed by the blood test center being registered in a data base equipped in the home doctor center, and initial diagnosis of the subscriber being made on the basis of the results of tests.

In the first aspect of the invention, subscriber's healthcare is possible by analyzing physical condition data of the subscriber, and a disease can be early diagnosed and early remedied by cooperation with medical institutions. In the second and third aspects of the invention, healthcare, early diagnosis and early remedy are possible by the use of blood test data of the subscriber, similarly to the first aspect.

According to the fourth aspect of the invention, there is provided a blood capsule comprising: a plurality of capsules connected by hollow pipes; and a seal portion for sealing the interior of the hollow pipes and the capsules in a reduced pressure condition, wherein a needle of a blood-collecting injection appliance penetrates the seal portion and introduces blood into the plurality of capsules, and wherein the capsules are cut and separated into discrete capsules and set in a blood test apparatus.

The capsule according to the fourth aspect of the invention can collect only a necessary amount of blood and can improve the blood test speed.

According to the fifth aspect of the invention, there is provided an injection appliance for injection or collection of blood comprising: an enlarged opening at a tip end of a needle. According to the sixth aspect of the invention, there is provided an injection appliance for injection or collection of blood comprising: a stopper located at a predetermined distance from a tip end of a needle. According to the seventh aspect of the invention, there is provided an injection appliance for injection or collection of blood comprising: a plurality of needles. According to the eighth aspect of the invention, there is provided an injection appliance for injection or collection of blood comprising: a needle unit and a main body or a capsule connected by a flexible tube.

The fifth and seventh aspects of the invention enables to increase the amount of drug solution that can be injected per unit time or enables to increase the amount of blood that can be collected per unit time, and can decrease the time for injection and collection of blood. The sixth aspect of the invention can prevent a needle sticks deep under skin. In the eighth aspect of the invention, since the needle and the main body are separated, it is prevented that movements of a physician or nurse are transmitted to the needle in a stuck condition.

### BRIEF DESCRIPTION OF THE DRAWINGS

FIG. 1 is a block diagram that shows outline of an entire system according to an embodiment of the invention;

FIG. 2 is a block diagram for explaining functions of a home doctor center in the embodiment of the invention;

FIG. 3 is a block diagram that mainly shows flows of money in the embodiment of the invention;

FIG. 4 is a block diagram that shows outline of an entire system according to a further embodiment of the invention;

FIG. 5 is a schematic diagram for explaining registration of an application and collection of blood at home in the further embodiment of the invention;

FIG. 6 is a schematic diagram for explaining collection of blood at a blood stand and payment to a blood test center in the further embodiment of the invention;

FIG. 7 is a schematic diagram for explaining a report of a result of primary diagnosis in the further embodiment of the invention;

FIGS. 8A through 8C are cross-sectional views for explaining the first example of blood-collecting injection appliance usable in the present invention;

FIG. 9 is a cross-sectional view for explaining an example of blood capsule usable in the invention;

FIGS. 10A and 10B are schematic diagrams for explaining the second example of injection appliance usable in the invention;

FIGS. 11A and 11B are schematic diagrams for explaining the third example of injection appliance usable in the invention;

FIGS. 12A and 12B are schematic diagrams for explaining the fourth example of injection appliance usable in the invention;

FIGS. 13A through 13D are schematic diagrams for explaining the fifth example of injection appliance usable in the invention;

FIGS. 14A and 14B are schematic diagrams for explaining the sixth example of injection appliance usable in the invention;

FIGS. 15A and 15B are schematic diagrams for explaining the seventh example of injection appliance usable in the invention;

FIGS. 16A and 16B are schematic diagrams for conceptionally explaining one and another examples of blood-collecting units usable in the invention; and

FIGS. 17A and 17B are schematic diagrams for concretely explaining one and another examples of blood-collecting units usable in the invention.

### DETAILED DESCRIPTION OF THE PREFERRED EMBODIMENTS

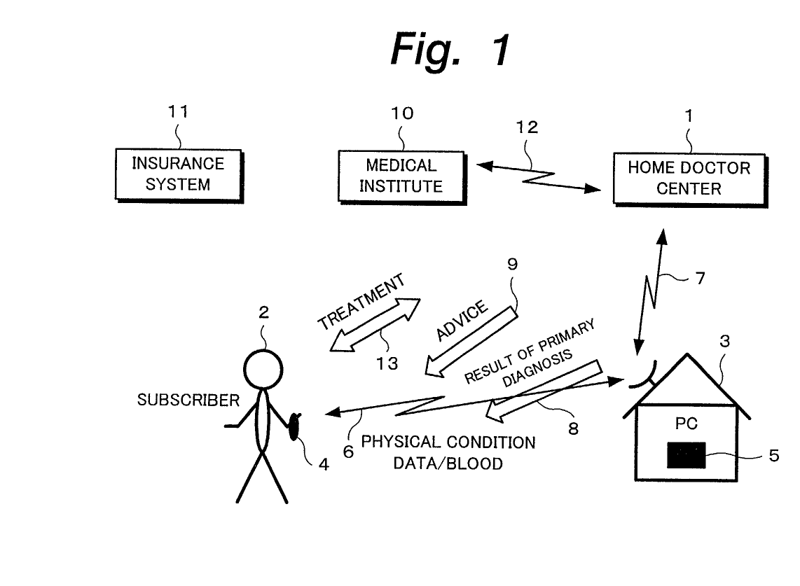

Explained below is an embodiment of the invention. First referring to FIG. 1, system configuration is explained. Numeral 1 denotes a home doctor center that is the central part of a home doctor system. Numeral 2 denotes a subscriber of the home doctor system, and numeral 3 denotes the subscriber's residence. The subscriber 2 carries a portable (mobile) physical condition-monitoring device 4. The residence 3 is equipped with a desktop or other type personal computer 5, for example.

The portable physical condition monitoring device 4 and the personal computer 5 at the residence 3 are connected by a wireless communication path 6 for one-way or two-way communication, and physical condition data measured by the physical monitoring device 4 is transmitted to the personal computer 5. Between the residence 3 and the home doctor center 1, a contact path 7 such as wired or wireless communication path, mail service or special collection and delivery system, for example, is provided. Internet is an example of wireless contact path 7. Through the contact path 7, the subscriber's physical condition data is transmitted to the home doctor center 1. Additionally, blood collected from the subscriber 2 at the residence 3 is sent to the home doctor center 1 via the contact path 7.

The home doctor center 1 receives the physical condition data and the blood sent from the subscriber. In the home doctor center 1, the physical condition data is analyzed by using software for initial (primary) diagnosis, and the received blood is tested by using an automated blood testing system. Further, subscriber's DNA is decoded and stored as a database. Additionally, the name of any hereditary disease identified by DNA decoding and analysis is additionally included in the database. Based on the results of analysis of the physical condition data and the result of the blood test, primary diagnosis of the subscriber is done. The result of the primary diagnosis (also using terms of primary diagnosis results adequately) 8 and advice 9 based thereon are notified to the subscriber 2 through the contact path 7 or other communication means.

The home doctor center 1 has a communication path 12 for contact between a medical institute 10 and an insurance system 11. The insurance system 11 is an administrative entity such as life insurance company, healthcare insurance association, or the like. Information on the subscriber 2, such as physical condition data and others, is given to the medical institution 10 such that the subscriber 2 can undergo adequate medical treatment 13. Further, physical condition data, and others, are given to the insurance system 11 dealing with life insurance and healthcare insurance, for example. A life insurance company belonging to the insurance system 11 calculates insurance premium the subscriber should pay, on the basis of the received physical condition data and others.

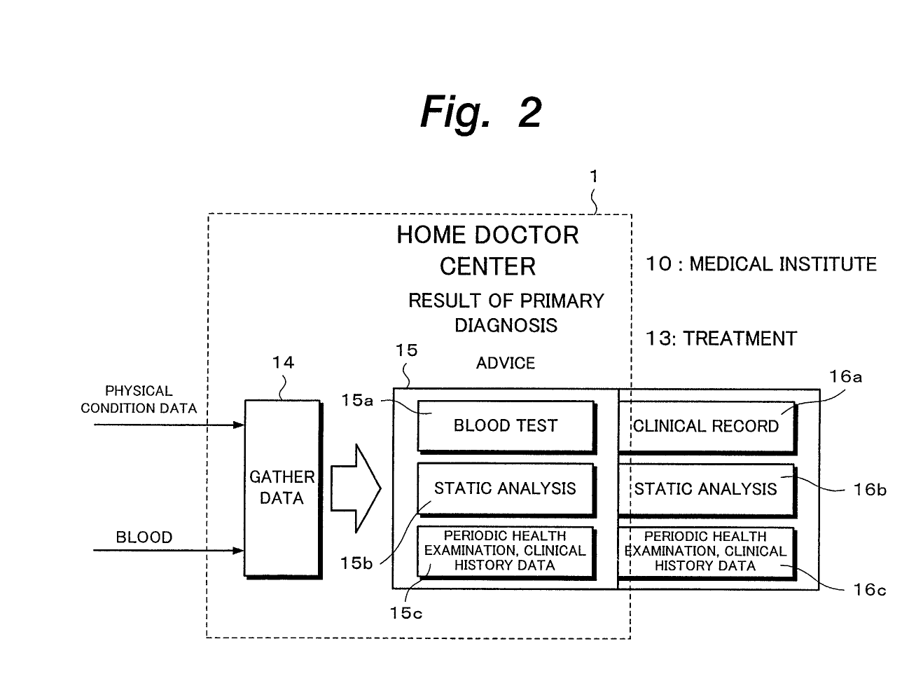

The above-mentioned home doctor system is explained below in greater detail. First explained are functions of the home doctor center 1, with reference to FIG. 2. The home doctor center 1 has the function 15 of receiving blood and physical condition data from the subscriber 2 through a data collecting section 14 and diagnosing physical condition of the subscriber 2. The home doctor center 1 has an automatic blood testing system that can conduct blood test of an enormous number of subscribers in a frequency about once a week or once a month.

Further, the home doctor center 1 has subscriber database, not shown. The subscriber database stores information about clinical histories, results of periodic health examinations and results of initial diagnoses of subscribers. The primary diagnosis function 15 is to initially diagnosing physical condition of a subscriber by using software from the result of the blood test 15a, static analysis 15b of the blood test result 15a and the physical condition data, and periodic health examinations and clinical history data 15c of the subscriber in the database. The center 1 has the function of developing the software as well. The result 8 of the initial diagnosis, dietary menu based on the result of the initial diagnosis, menu for building up the physical strength and advise 9 about health care are informed to the subscriber 2.

The home doctor center 1 also has the function of connecting the subscriber 2 and the medical institution 10, if the subscriber is currently a patient of the medical institution 10, upon monitoring physical condition during home care or upon any change in physical condition, by informing the medical institution 10. The medical institution 10 reflects the results of the initial diagnosis received from the home doctor center 1 to the clinical record 16a, static analysis 16b and data base 16c of periodic health examinations and clinical history. In this manner, when the subscriber undergoes medical treatment in the medical institution 10, information about the initial diagnosis is delivered to the medical institution 10 as a basic data of the subscriber, and early diagnosis and early remedy of a subscriber's disease are possible.

Next explained measurement and collection of physical condition data. Explained below is a specific example of the portable physical condition-monitoring device 4. Its functions can be generally divided into physical condition monitoring for dietary and physical strength improving purposes and healthcare monitoring. Healthcare monitoring is conducted for early diagnosis and early remedy of a disease.

As items of physical condition monitoring, there are heart rate, blood pressure and consumed calories. Used as a device is a portable consumed calories measuring device. This device is configured to measure consumed calories by measuring the number of steps and walking speed of a subscriber. That is, it calculates the walking speed from the number of steps per unit time, and measures consumed calories by (velocity²xk (k: coefficient)). The number of steps can be measured in the same manner as conventional pedometers. Regarding its carrying mode, there are the type attached to a waist belt, the type held around an ankle, the type held on a wrist, the type incorporated in a wristwatch, and others.

By adding the function of measuring pulses to the portable consumed calories measuring device, early diagnosis of physical strength, physical exhaustion and physical condition is possible. This relies on that a person with a higher basic physical strength exhibits less increase of pulses per unit-consumed calories. By further adding the functions of measuring pulses and blood pressure to the portable consumed calories measuring device, initial diagnosis of physical strength, physical exhaustion and physical condition is possible from the time of exercise and amount of exercise versus changes in pulses and blood pressure with time.

The portable consumed calories measuring device has a communication function, and various measure data (data on consumed calories, pulses and blood pressure) are transmitted as physical condition data to the residence 3 via the wireless communication path 6, and taken into the personal computer 5 through a receiver device (such as antenna, for example) prepared in the residence 3. In this case, it is also possible to transmit measured data directly to the home doctor center 1. For transmitting and receiving physical condition data, it is possible to temporarily store data in memory of the monitoring device and reading data in the monitoring device from the part of the home doctor center 1 or the residence 3. In this case, particular read-out devices may be installed at stations, or the like, such that subscribers can transmit data through the read-out device. Alternatively, portable telephones or other like devices may be used for transmitting data.

A concrete example of the portable physical condition-monitoring device 4 for the health care purpose is a wireless electrocardiogram-measuring device. It includes a potential detector having an electrode for detecting weak potentials generated upon contraction and expansion of a heart and a transmitter for modulating and transmitting detected potentials, and a transmitter/receiver for receiving an electric wave from the potential detector, then converting it into electrocardiogram data and externally transmitting the electrocardiogram data. The potential detector is a wrist-belt type held on a subscriber's wrist, or an ankle-belt type held on a subscriber's ankle. For reliably obtaining potential difference between electrodes in a wireless system, a basic waveform is used as the transmission waveform, and an inverter type is employed.

By using such a wireless structure, it is possible to remove the problems of conventional electrocardiogram measuring devices whose detectors and control power sources are controlled by cables, i.e., connection of the detector to a body being troublesome, the need for a long time to measurement, and inconvenience for portable use.

A concrete example of portable physical condition monitoring device 4 for the health care purpose is a laser type hemoglobin-measuring device. It comprises a detector having a green laser emitter and a photo detector, reflectance operator for comparing amount of emitted light with amount of detected light, modulator/transmitter for modulating and transmitting the reflectance, and receiver/converter for receiving information from the modulator/transmitter and converting it into amount of hemoglobin. Since hemoglobin largely changes in reflectance against green light as compared with leukocytes and erythrocytes in blood, changes in reflectance can be converted to and measured as amount of hemoglobin. Unlike blood test, the laser type hemoglobin measuring device may be operated also by operators other than qualified physicians or nurses to perform measurement any time, and it is convenient for portable use.

The wireless electrocardiogram measuring device and the laser type hemoglobin measuring device also include communication function as explained concerning the portable consumed calories measuring device, and measured data can be sent to the home doctor center 1 or the personal computer in the residence 3.

It is also possible to have urine sent from the subscriber 2 to the home doctor 1 in addition to blood and test the urine and use its result for initial diagnosis.

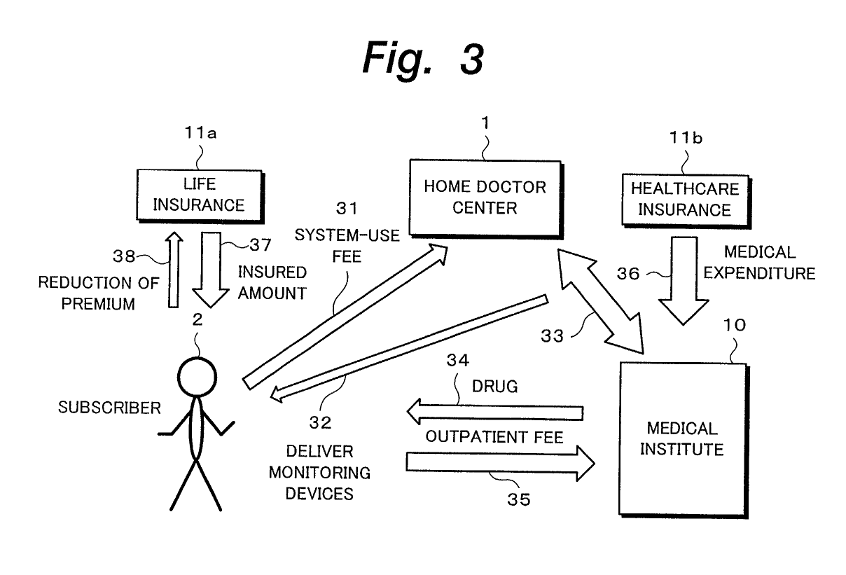

As explained above, the home doctor 1 manages overall data about health of the subscriber 2. Not limited to this, the home doctor 1 also has the function of totally managing medical expenditure and insurance premium of the subscriber 2. FIG. 3 shows the home doctor system, focusing at the flow of money.

Between the home doctor center 1 and the subscriber, the subscriber 2 pays the fee for using the system (path 31), and the home doctor center 1 distributes monitoring devices to the subscriber (path 32). The fee may be paid in various modes, such as each month, each year or each time when the subscriber 2 receives a result of diagnosis. Further, the home doctor center land the medial institution 10 are in business tie-up (path 33), the medical institution 10 delivers drugs to the subscriber 2 (path 34), and the subscriber 2 pays outpatient clinical charges (path 35). Clinical charges are partly paid by the subscriber 2 and partly paid by a healthcare insurance 11b, a part of the insurance system 11, to the medical institution 10 (path 36).

Between a life insurance 11a, a part of the insurance system, and the subscriber 2, payment of insurance premium (path 38) and payment of insured amount (path 37) may occur. Since the home doctor center 1 is in business tie-up with the insurance system 11, a life insurance company belonging to the insurance system 11 can receive advice given to the subscriber 2, subscribers data, and soon, and can calculate reasonable premium of the subscriber.

That is, the life insurance company can calculates risks of outbreaks of diseases and death from the state of health of the subscriber 2, and premium the subscriber 2 can be calculated. In this case, the premium may be changed depending upon the risks. This insurance system can precisely grasp the state of health of the subscriber 2 as compared with conventional systems taking the state of health only at the time of subscription into consideration for calculating the premium, and the premium the subscriber 2 should pay can be reduced. The same applies not only to life insurance but also to accident insurance and medical insurance.

In the above-explained home doctor system, it is necessary to prevent violation of the subscriber's privacy. For example, for enhancing secrecy of data about each subscriber, transmitted, received and stored subscriber data are encoded.

In the system shown in FIG. 1, the subscriber collects his blood at home, and sends it to the home doctor 1 such that blood test and initial diagnosis are conducted there for the subscriber 2. This is called a blood testing system. For this purpose, it is necessary to prepare a blood collecting injection unit with which the subscriber can collect blood by himself or herself. The blood collecting injection unit is configured to collect minimum blood enough for initial diagnosis within the extent of non-medical activity. Concrete examples of injectors usable for blood collection will be explained later.

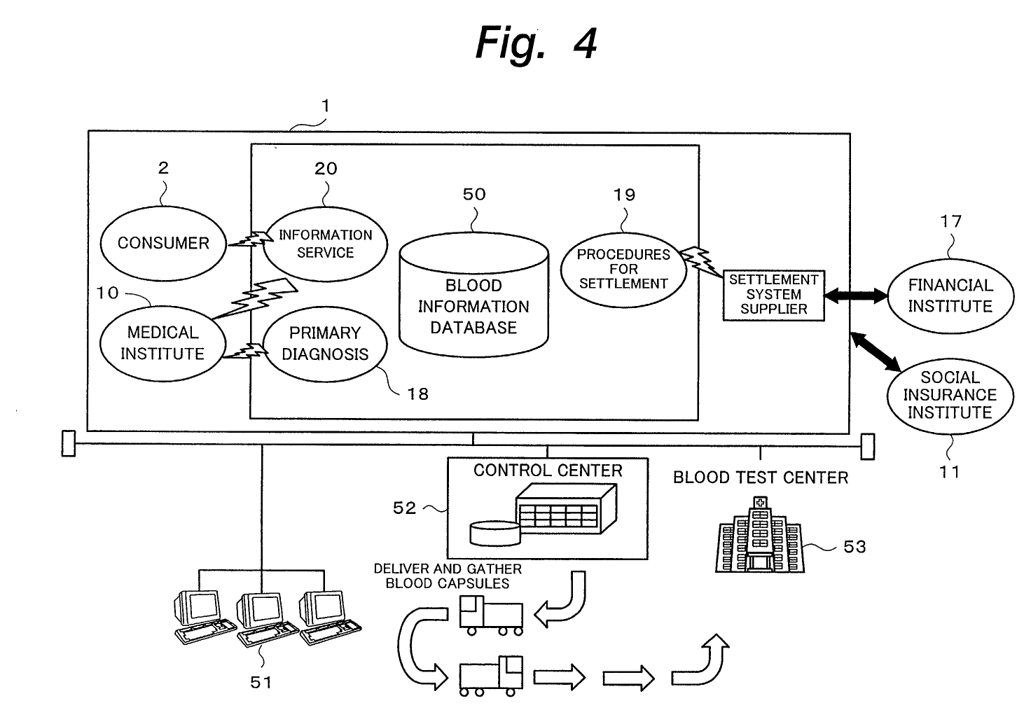

FIG. 4 shows another embodiment of the home doctor system having a blood testing system as its core. In this embodiment, physical condition monitoring data is treated as an optional material, and the home doctor center 1 is designed to execute primary diagnosis based on a data base and a result of blood test, enabling blood collection in places other than subscribers' residences, and entrusting blood tests to external testing centers other than the home doctor center 1. For example, blood-collecting stands are prepared at readily accessible locations like stations, drug stores, or the like, such that nurses can collect blood at blood-collecting stands, or alternatively, nurses visit subscriber's residences and collect blood there.

The home doctor center 1 has a blood information database 50. Numeral 51 denotes a blood-collecting site (blood-collecting stand, residence, or the like), 52 denotes a control center for management of blood collection, and 53 denotes a blood test center. The home doctor center, these blood-collecting stand 51, control center 52 and blood test center 53 are connected to each other by a network, mail service or other means.

The control center 52 manages operation of the delivery and collecting system. The delivery and collecting system delivers blood-collecting capsules to the blood-collecting site 51, and collects blood-collecting capsules containing collected blood. The collected blood-collecting capsules are brought into the blood test center 53, and undergo blood tests. Results of blood test are sent to the home doctor center 1 via a network, for example, and stored in blood information database 50.

The home doctor center 1 has primary diagnostic function 18, settlement procedure function 19 and information service function 20. The information service function 20 is a service of giving various kinds of information to the subscriber, such as notice on the result of primary diagnosis. Additionally, based on the result of primary diagnosis, it gives subscribers additional information. Additional information includes information useful for subscribers to prevent and cure diseases.

More specifically, depending on the result of primary diagnosis, details and maps of locations of nearest medical institutions, pharmacies and healthcare food shops that are registered in the home doctor center 1, as well as other information, is given to the subscriber 2. It is also possible to give maps of blood-collecting stands, and their states of congestion and other information. In this case, gathering a registration fee from each medical institution 10 (or free of charge), information about medical institutes to the extent without propaganda effect is given free of charge to the subscriber. Information about pharmaceutical companies, pharmacies and healthcare food shops is given free of charge to the subscriber, gathering registration fees and advertising costs from these entities. Further, based on primary diagnosis, a medical prescription may be made, and according to that, drugs and healthcare food may be sold through Internet. In this case, the settlement procedure function 19 is used for payment, and purchased goods may be delivered by using the blood capsule delivery system.

With the above-explained information service function 20, the subscriber (consumer) 2 can readily find out the nearest medical institution when having a result of primary diagnosis or any subjective symptom, and can readily find out pharmacies, healthcare food shops, sports gyms, and so on. Furthermore, collection of registration fees and advertising expenses enable free information services to subscribers.

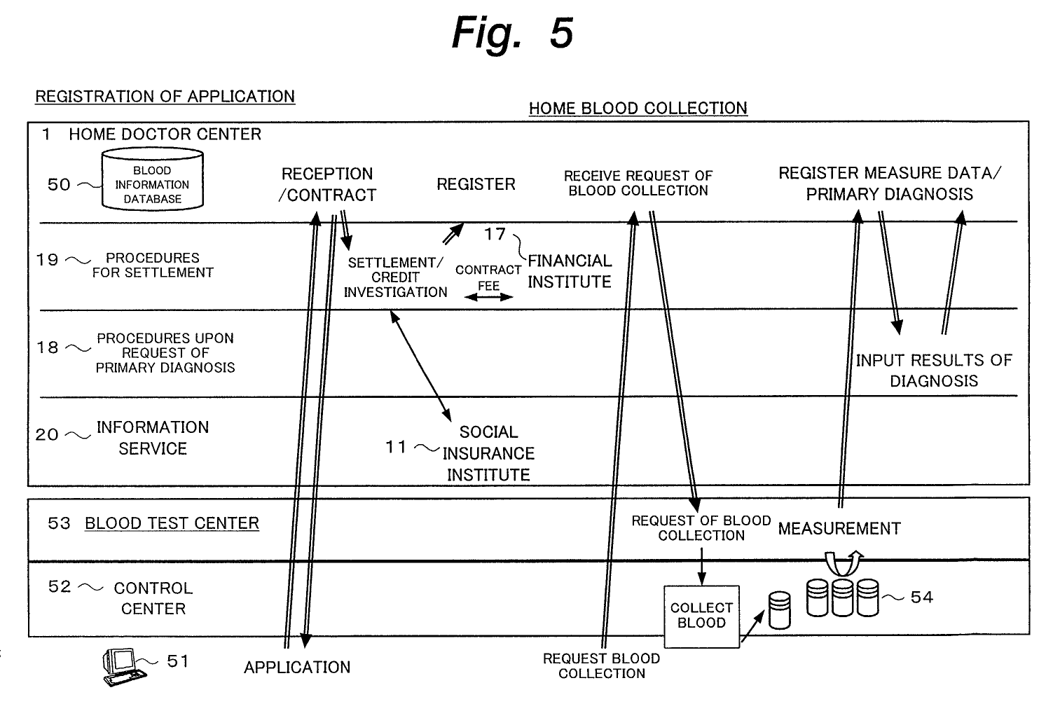

The home doctor system shown in FIG. 4 is explained below in greater detail with reference to FIGS. 5, 6 and 7. In these figures, a flow of time is shown in the horizontal direction, and functions of the home doctor center 1 are shown in the vertical direction. FIG. 5 shows procedures responsive to a request of subscription from a consumer who came to the blood-collecting site 51. This request is done by writing the subscriber's name, address, date of birth, information on his credit card, serial number of his healthcare insurance, and so forth. The home doctor center 1 receives this request through a network or a mail service.

The home doctor center 1, after receipt of the request, cooperates with a financial institution to conduct credit administration, for example, and requests information about the healthcare insurance to a social insurance institute 11. If it obtains acceptable answers, it registers the subscriber in the blood information database 50 and issues ID to the subscriber. Through these procedures, registration is completed.

In case of blood collection at home (home blood collection), a request for collecting blood occurs in response to entry of ID. The blood collection request is acknowledged by the home doctor center 1. Since the system is prepared to send nurses or other qualified persons to subscribers' residences to collect blood there, the home doctor center 1 instructs the control center 52 to visit the residence and collect blood there and simultaneously issues a capsule serial number. Pursuant to the instruction, the control center 52 works for collection of blood. More specifically, it determines the schedule of the visit. Optionally, tests of other materials of physical condition, such as pulses, blood pressure, urine and temperature, for example, are prepared upon blood collection.

The capsule resulting from blood collection is sent to the blood test center 53. The blood test center 53 executes a blood test (measurement) and sends the result of the test identified by the capsule serial number to the home doctor center 1. The home doctor center 1 registers the measurement data to the database 50. Additionally, when an instruction of primary diagnosis is issued, a physician conducts primary diagnosis from the measurement data, and the result of the diagnosis is input.

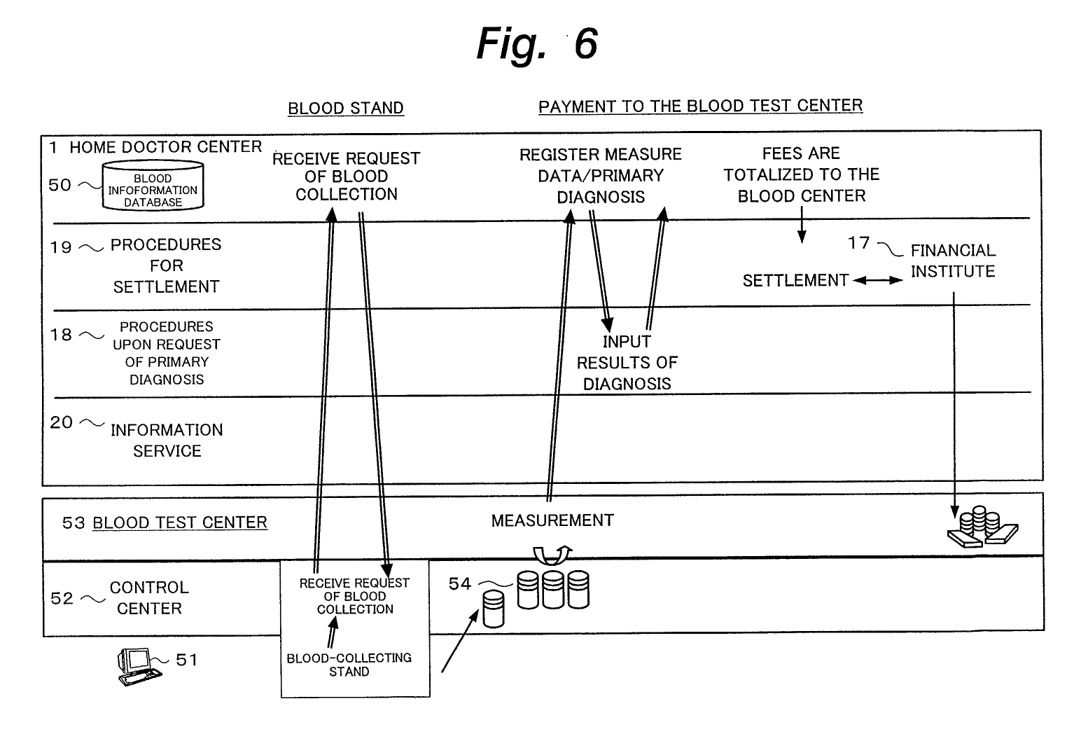

FIG. 6 shows a flow of procedures upon blood collection at a blood-collecting stand as a blood-collecting site 51. The blood-collecting stand is under management of the control center 52. An application for blood collection, which was received at the blood-collecting stand, is sent to the home doctor center 1. The received application contains ID. If the home doctor center 1 judges this application acceptable, it issues a capsule serial number and sends an instruction to a nurse at the blood-collecting stand to execute blood collection. Alternatively, at the blood-collecting stand, the subscriber merely applies for and fixes reservation for blood collection at his residence on another day.

The capsule containing blood collected at the blood-collecting stand is gathered by the control center 52 and transported to the blood test center 53. The blood test center 53 executed a blood test. The result of the measurement is sent to the home doctor center 1, and registered in the blood information database 50. In accordance with a diagnosis instruction, a physician conducts primary diagnosis from the measurement data, and the result of the diagnosis is input. For example, the result of the diagnosis is registered in the blood information database 50.

Since the home doctor center 1 and the blood test center 53 are independent systems from each other, the home doctor center 1 has to pay fees for the test to the blood test center 53. Therefore, fees are totalized, and the test fees are settled through the financial institute 17. For example, test fees corresponding to measured items and measured cases are paid every month, for example, from the home doctor center 1 to the blood test center 53.

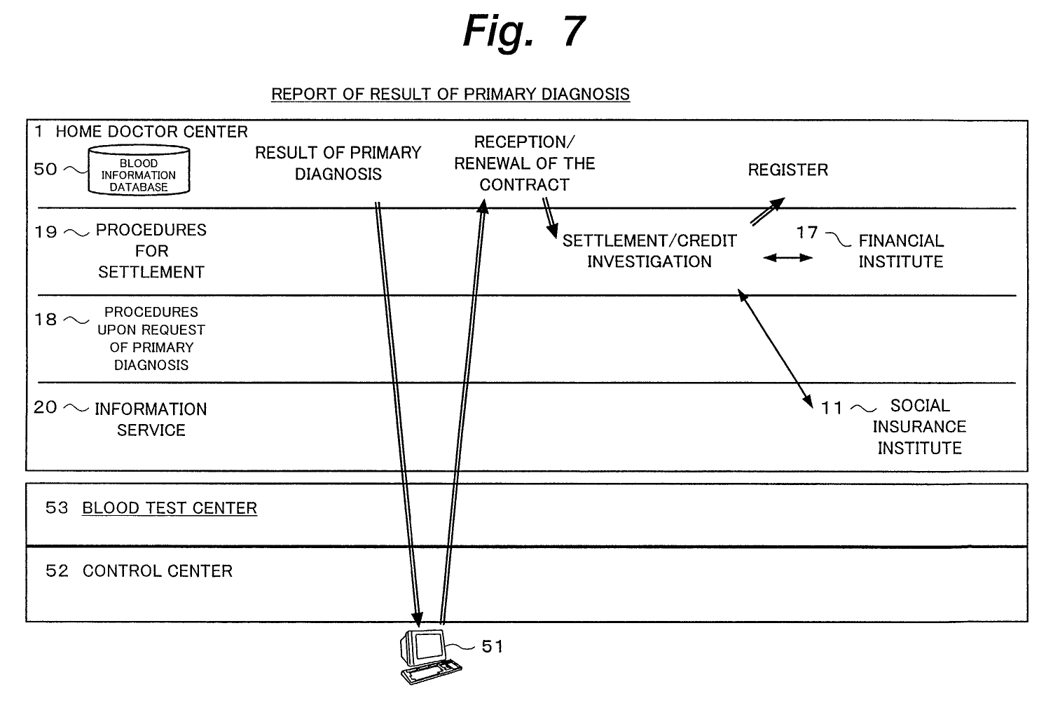

FIG. 7 shows procedures of reporting the result of primary diagnosis. The result of primary diagnosis is reported from the home doctor center 1 to the subscriber 2 through a network, mail service or other means. The subscriber is also allowed to request renewal to the home doctor center 1, fixing test items, frequencies, term of the contract, and so forth. In receipt of the request for renewal of the contract, the home doctor center 1 cooperates with the social insurance institute 11 and the financial institute 17 for credit investigation, and renews the contract if the result of the research is acceptable.

In one and the further embodiments of the above-explained home doctor system, primary diagnosis is based on the result of testing collected blood and physical condition data. For the purpose of daily control of physical condition and diagnosis, blood is collected periodically. Therefore, loads to subscribers caused by blood collection (required time, pains, etc.) are preferably minimized.

Explained below are specific examples of blood-collecting units usable for blood collection according to one and the further embodiments of the invention. Explanation made below is forwarded, centered on blood collection. However, they are equally applicable also to injection.

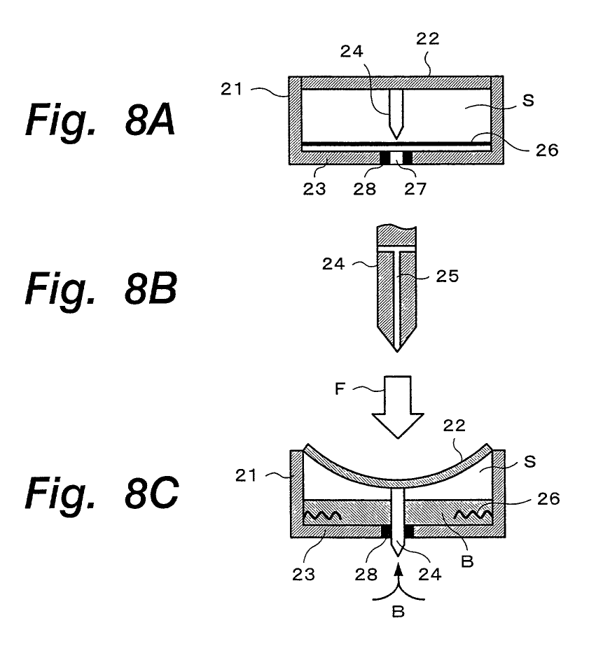

FIGS. 8A through 8C show an example of blood-collecting injection appliance. Numeral 21 denotes a sidewall member of a case (made of plastic resin, for example) of the injection appliance, and the sidewall member 21 has a cylindrical shape, for example. Numeral 22 denotes a flexible operation plate 22 covering one surface of the sidewall member 22. Numeral 23 denotes a contact plate that is integral with the sidewall member 21. A metal needle 24 projects from about the center of the operation plane 22 toward the contact portion 23. As shown in FIG. 8B, the needle 24 is divided horizontally from its tip end toward the root end to define a suction slit 25 in communication with the outside.

As shown in FIG. 8A, the tip end of the needle 24 has a length to reach slightly inward of the contact plate 23 of the case. Between the tip end of the needle 24 and the contact plate 23, a thin film 26 is provided to partition the interior space of the case. The contact plate 23 has formed a hole 27 having a diameter slightly smaller than that of the needle 24 at the location aligned with the tip end of the needle 24. A packing member 28 is attached on the wall surface of the hole 27. The space S closed by the sidewall member 21, operation plate 22 and thin film 26 is held in a vacuum or in a highly reduced pressure.

As shown in FIG. 8C, the subscriber 2 puts the injection appliance on a part of his body sensing less pains, such as ear lobe while bringing the contact plate 23 into contact 23 with it, and applies a force F onto the operation plate 22. As a result, the operation plate 22 deforms toward the contact plate 23 and the tip end of the needle 24 penetrates the thin film 26 and slightly stuck into the subscriber's body. Since the space S is held in a vacuum or a highly reduced pressure, blood B of the subscriber is pumped up into the space S through the suction slit 25 at the tip end portion of the needle 24. Even after the force F is removed, the operation plate 22 is held deformed, and prevents leakage of the blood B pumped up.

After collection of blood, since the tip end of the needle 24 slightly projects externally, blood is packed in a capsule for the transport purpose. The capsule bears a seal of a capsule serial number assigned to the subscriber 2. The blood test center 53 draws out the blood from the capsule and executes blood test. Blood is drawn out from the injection appliance by a mechanism attached to a blood test apparatus. A main blood test is an immunological test.

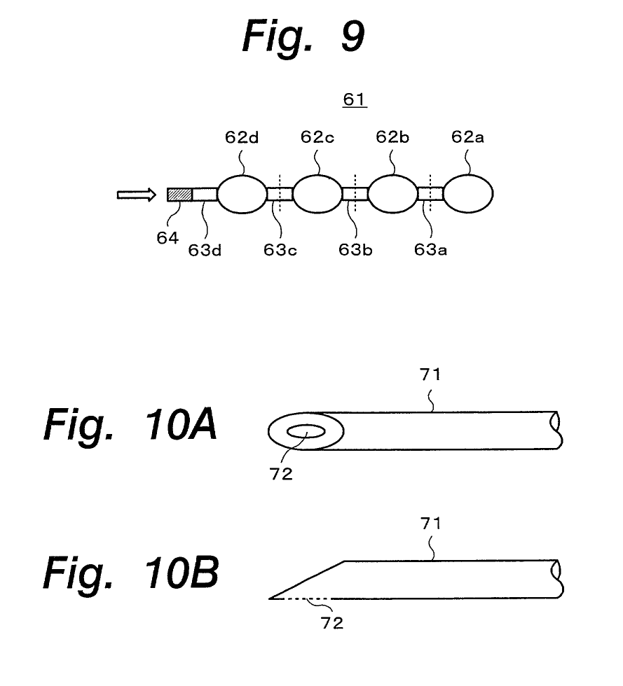

FIG. 9 shows an example of capsule used for transporting collected blood. In FIG. 9, numeral 61 denotes the capsule that includes a plurality of capsules 62a, 62b, 62c and 62d coupled by hollow pipes 63a, 63b and 63c. A number of capsules corresponding to the number of blood test items are connected in series. Shapes and sizes of the capsules are determined in accordance with the test apparatus, test method and other requirements. A pipe 63d extending from the capsule 62d at one extreme end, and the outer end of the pipe 63d has a seal portion 64. Interior space of the capsules and pipes closed by the seal portion 64 is previously evacuated. The capsule 61 is made of a material (resin, for example) hard enough to define the interior space in the capsule even after evacuation.

After collection of blood, the tip end of the needle of the blood-collecting injection appliance is stuck through the seal portion 64. Since the interior space of the capsule 61 is held in a reduced pressure, blood flows from the blood-collecting injection appliance into the capsules 62a through 62d through the hollow pipes 63a through 63d. The seal portion 64 is made of a material that automatically close the pierced hole so as to prevent leakage of blood after removal of the needle. The capsule containing the blood is sent in the serially coupled form to the blood test center 53. The blood test center 53 cuts the capsule into discrete capsules 62a through 62d as shown in FIG. 9 by using a cutter having a laser or a heater, and sets them on a test apparatus. Then, tests (measurements) are conducted with blood in the capsules.

Since the above-explained capsule 61 can contain an amount of blood just enough for intended tests and can be separated into discrete capsules that can be set on a test apparatus, it is possible to prevent collection of an excessive amount of blood and to remove the process of dividing blood into parts for different test items after collection. Therefore, tests can be speeded up and readily automated.

FIGS. 10A and 10B show an example of blood-collecting needle intended to alleviate the load during blood collection. FIG. 10A is a view of the tip end of a needle 71, taken from above. FIG. 10B is a view of the needle 71 taken from one side. The needle 71 has a hole 72 at its tip end for introducing blood. The hole 72 facilitates inflow of blood and outflow of a parenteral solution, and enables collection blood from a peripheral vessel and injection of a parenteral solution into a layer under the skin in a short time. As a result, load during blood collection or injection can be reduced.

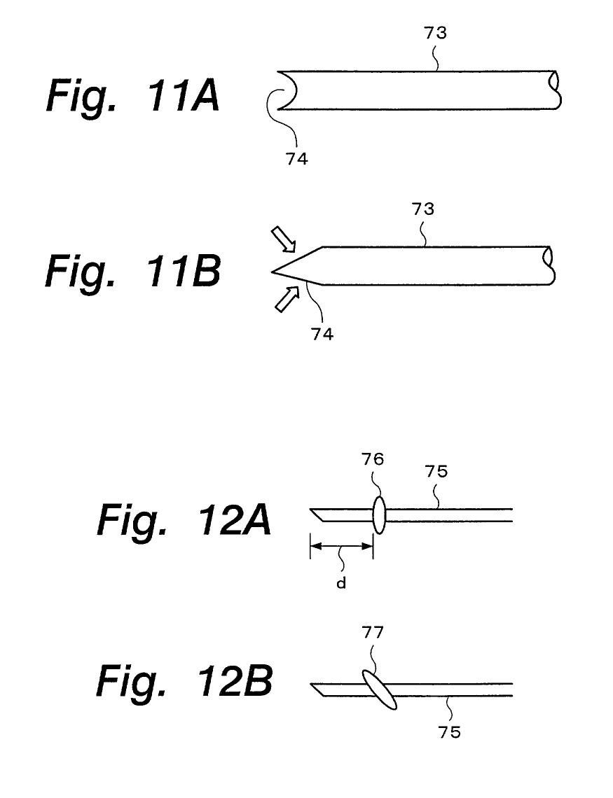

FIGS. 11A and 11B show another example of blood-collecting needle. FIG. 11A is a view of the tip end of a needle 73 taken from above. FIG. 11B is a view of the needle 73 taken from one side. The needle 73 has a tip end opening 74 made by cutting opposite side portions upon cutting out the tip end diagonally. The tip end opening 74 enables inflow of blood and outflow of a drug solution from opposite sides as shown by arrows, and alleviates the load during blood collection or injection.

A needle 75 shown in FIG. 12A has a stopper 76 at a position distant by d from its tip end. As shown in FIG. 12B, the needle may have a stopper 77 angled from a line intersecting the needle 75. In this manner, the needle is prevented from sticking excessively deep. Value of d can be changed among adults, children, sexuality, degrees of obesity, and so on. For example, d may be 2 through 3 mm. Additionally, as shown in FIG. 12B, inclination of the stopper 77 ensures the function as the stopper even when the needle 75 is stuck diagonally.

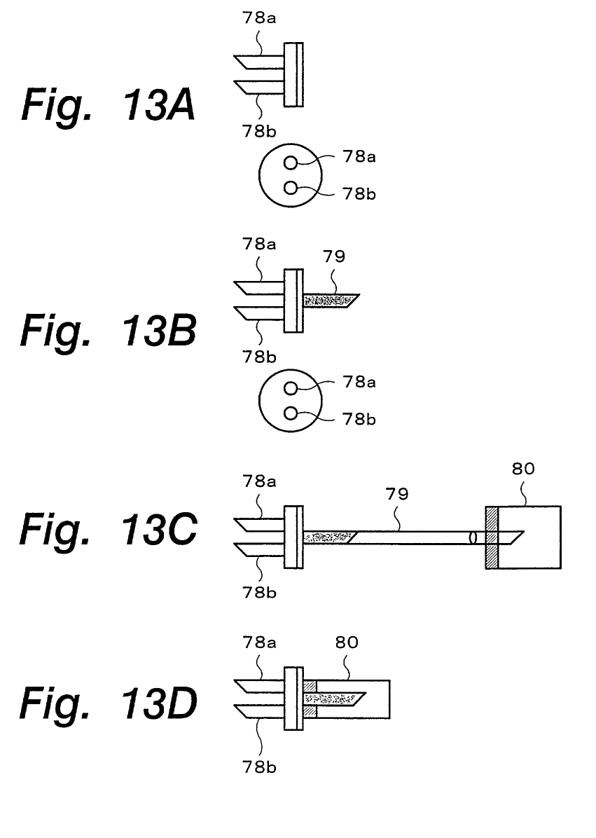

FIG. 13A shows an injection unit having a plurality of needles, e.g. two needles 78a and 78b. The injection unit has the same structure as that shown in FIGS. 8A through 8C. Having a plurality of needles, the injection unit enables blood collection and injection in a short time. Configuration of tip ends of the needles may be a combination of those explained with reference to FIGS. 10A, 10B, FIGS. 11A and 11B.

Instead of injection units configured to store collected blood or parenteral solution inside, a unit to be connected to an external means through a flexible tube 79 may be used as shown in FIG. 13B. More specifically, as shown in FIG. 13C, an injection unit having needles 78a and 78b is connected to a capsule 80 by a tube 79. The capsule 80 receives inflow of collected blood. Furthermore, as shown in FIG. 13D, it is also possible to connect the injection unit directly to the capsule 80. Not only blood collection but also injection of a parenteral solution is possible.

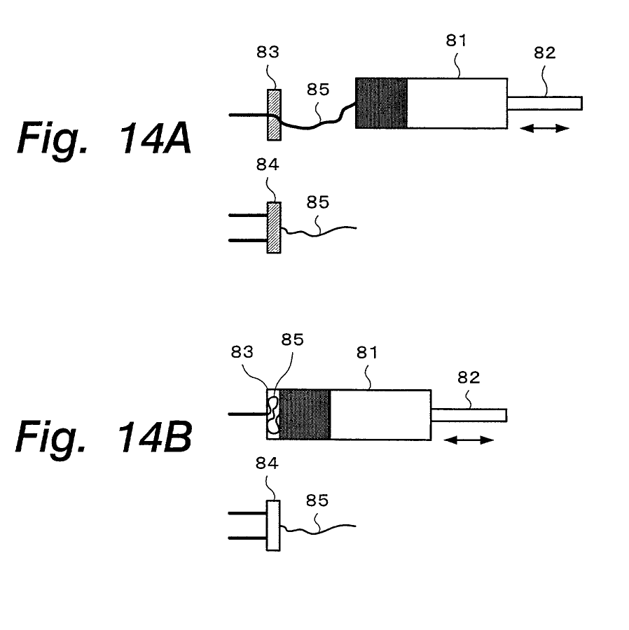

FIG. 14A shows an example of injection unit. An injection unit main body 81 is configured to draw blood into the main body 81 or send out a drug solution from the main body 81 by manipulation of a lever 82. Separately from the main body 81, a needle unit 83 is used. The needle unit 83 may have a single needle or a plurality of needles as shown in FIGS. 13A through 13D (shown at 84). The opening at the tip end of the main body 81 and the needle unit 83 or 84 are connected by a flexible tube 85. The tube 85 is detachably connected to the main body 81 and the needle unit 83 or 84.

As shown in FIG. 14B, it is also possible to employ the configuration in which the needle unit 83 can be attached to the injection unit main body 81. Alternatively, the needle unit 84 having a plurality of needles may be used. In the example of FIG. 14B, the needle unit 83 or 84 is detached after insertion of the needle or needles.

Since the injection units shown in FIGS. 14A and 14B are separated from main bodies, they can prevent transmission of movements from physician's or nurse's hands. Therefore, it is possible to remove pains caused by movements of the needle or needles stuck into a body.

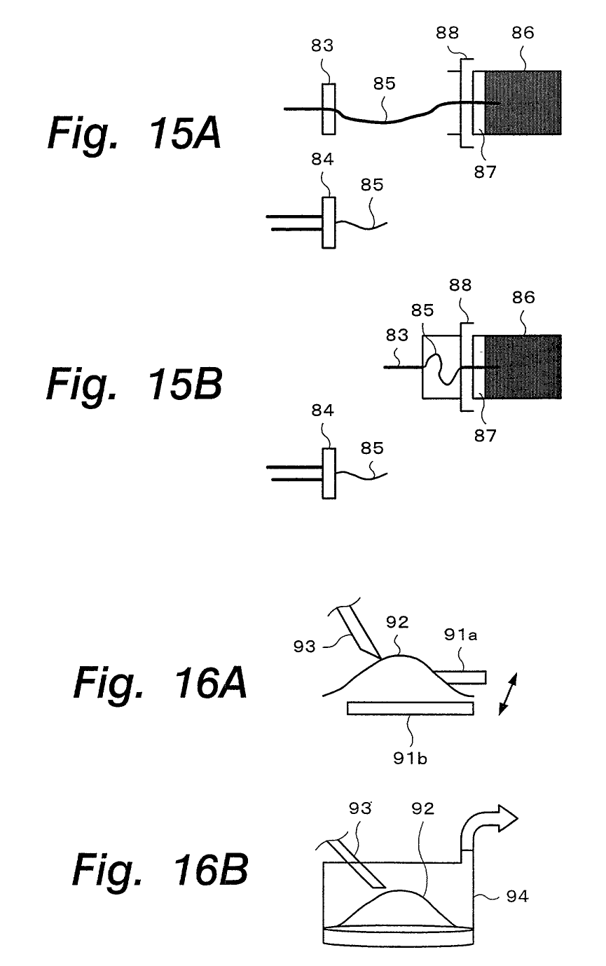

FIGS. 15A and 15B show an example of blood-collecting injection unit that prevents transmission of movements of hands to the needle unit. While the examples shown in FIGS. 14A and 14B are used as injectors, configuration of FIG. 15 is for use in blood collection. Therefore, a needle or needles are located at one side of the tube 85 not connected to the needle unit 83 or 84, and the needle or needles are stuck into the seal portion 87 of the blood-collecting capsule 86. The blood-collecting capsule 86 has the configuration in which a container made of glass or resin is closed with the seal portion 87 made of rubber or resin. Interior of the capsule is held in a reduced pressure, and blood can be automatically introduced into the capsule when the needle or needles coupled to the tube are stuck into the seal portion 87.

Numeral 88 denotes a blood-collecting unit. The blood-collecting unit 88 is an auxiliary unit for ensuring the needle or needles to be reliably stuck into the region of peripheral vessels under the skin and for minimizing pains. Concrete configuration of the blood-collecting unit 88 will be explained later. In the configuration of FIG. 15A, the needle unit 83 and the blood-collecting capsule 86 are always separated. FIG. 15B shows a configuration in which the needle unit is integrally coupled with the blood-collecting unit 88 when it is stuck into the skin upon blood collection, but once it is stuck into the skin, the needle unit 83 or 84, tube 85 and blood-collecting unit 88 are separated from the capsule 86.

Blood-collecting units are explained below. FIGS. 16A and 16B show basic structures of blood-colleting units. FIG. 16A shows a blood-collecting unit configured to pinch skin 92 between movable portions 91a and 91b such that a blood-collecting needle 93 is stuck into the fixed, pinched-up skin 92. FIG. 16B shows a configuration in which skin 92 in a region where the needle 93 is intended to stick is covered with a sucking unit 94, and by reducing the pressure inside the sucking unit 94, the skin 92 is fixed and raised. Depth and angle of insertion of the needle can be adjusted when these blood-collecting units.

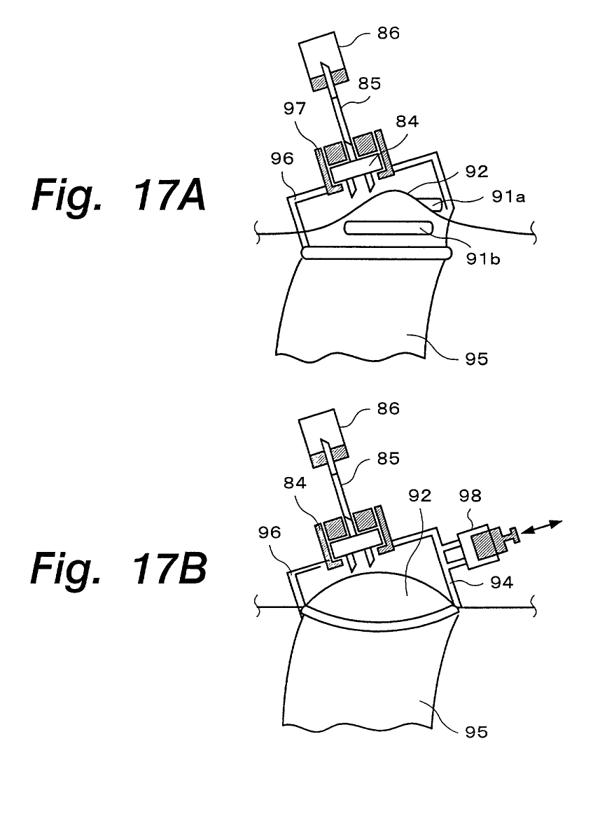

FIGS. 17A and 17B show concrete structures of blood-collecting units. They are fixed on persons by band belts wound around upper arms, for example. In the configuration shown in FIG. 17A, skin 92 is fixed by the movable portions 91a and 91b attached to a belt 95, and in this status, the needle of the blood-collecting injection unit is stuck. A blood-collecting unit has a frame 96 is equipped with a frame 96 for using the blood-collecting injection unit. The blood-collecting injection unit has the configuration explained with reference to FIGS. 15A and 15B, in which the needle unit 84 and the capsule 86 are connected by the tube 85. The needle unit 84 can slide inside a cylindrical stopper 97, and controlled to stick the needle to an appropriate depth.

FIG. 17B shows a concrete example of suction-type blood-collecting unit. The frame 96 is fixed at a desired position by the belt 95. The frame 96 defines a sealed space. The space in the frame 96 is reduced in pressure by using an evacuating cylinder 98. As a result, skin 92 is raised and fixed. The needle of the needle unit 84 is stuck into the skin 92. In this case, the needle unit 84 may be fixed at the lowered position to raise the skin 92 upward with the aid of the evacuating cylinder 98 such that the needle sticks to the raised skin 92.

As apparent from the foregoing explanation, according to the invention, by analyzing physical condition data of a subscriber or testing blood, subscribers daily health care can be managed, and by close tip-up with a medical institute, diseases can be early found and cured. Furthermore, according to the invention, by using a result of initial diagnosis for calculation of premiums, premiums can be reduced substantially.

According to the invention, a capsule for containing collected blood is in form of a plurality of capsules of a number, shape and size particularly determined for test items and test apparatus, for example, which are connected together and are cut into discrete capsules and set on the test apparatus upon actual blood tests. Therefore, it is prevented to collect blood beyond a truly necessary amount, and blood tests can be speeded up.

Additionally, injectors according to the invention alleviate loads during injection or collection of blood. That is, by employing a configuration enlarging the opening area at the needle tip end or having a plurality of needles, collection of blood from peripheral vessels with a small flow of blood and injection of a drug solution in such peripheral vessels can be executed in a short time. Furthermore, the injectors limit the sticking depth of needles with the aid of a stopper, and therefore minimize pains. Moreover, since the needle portion and the injector main body or blood-collecting capsule are connected by a flexible tube, movements of physician's or nurse's hands are prevented from reaching the needle stuck in a body, and pains are therefore alleviated. Further, by the use of the blood-collecting unit, needles can be reliably stuck into skin without special skill of physicians or nurses.

What is claimed is:

1. A home doctor system characterized in that:
data can be transmitted between a home doctor center and a plurality of subscribers, and each subscriber measures his or her physical condition data with a physical monitoring device and send it to the home doctor center; and
the home doctor center makes initial diagnosis of the subscriber's physical condition from said physical condition data, and informs the subscriber of the result of the initial diagnosis.

2. A home doctor system characterized in that:
data can be transmitted between a home doctor center and a plurality of subscribers, and each subscriber send his or her blood to the home doctor center; and
the home doctor center executes blood tests of said blood, makes initial diagnosis of the subscriber from results of the blood tests, and inform the subscriber of a result of said initial diagnosis.

3. A home doctor system comprising a home doctor center, a blood test center and a blood-collecting site that are connected together,
said blood-collecting site sending blood to said blood test center; and
results of tests executed by said blood test center being registered in a data base equipped in said home doctor center, and initial diagnosis of the subscriber being made on the basis of said results of tests.

4. The home doctor system according to claim 3 wherein said blood-collecting site is a subscriber's residence or a blood-collecting stand.

5. The home doctor system according to claim 4 wherein said blood-collecting stand is placed in a station or other readily accessible location.

6. The home doctor system according to claim 3 further comprising a delivery/gathering system for delivering blood capsules to said blood-collecting site, and gathering capsules containing blood and sending them to said blood test center.

7. The home doctor system according to claim 3 wherein said home doctor center issues a predetermined code and/or number to said blood-collecting site when said home doctor center accepts a blood collection request from said blood-collecting site, and collected blood is identified by said predetermined code and/or number.

8. The home doctor system according to claim 3 wherein said home doctor center arranges a visit of a qualified person to a residence when said home doctor center accepts a blood collection request from said blood-collecting site.

9. The home doctor system according to claim 1, 2 or 3 wherein information obtained by said home doctor center is used for calculation of premiums.

10. The home doctor system according to claim 1 wherein said physical condition data includes at least subscriber's pulses and blood pressure.

11. The home doctor system according to claim 2 wherein a subscriber's gene is decoded, and decoded genetic information is stored as a data base to use it for predicting and preventing possible diseases of the subscriber and for treatment of such diseases.

12. The home doctor system according to claim 1, 2 or 3 wherein said home doctor center includes a database storing data on each subscriber.

13. The home doctor system according to claim 1, 2 or 3 wherein said physical condition data or said results of blood tests are processed by software for initial diagnosis.

14. The home doctor system according to claim 1, 2 or 3 wherein said home doctor center can inform a medical institute of a result of said initial diagnosis.

15. The home doctor system according to claim 1, 2 or 3 wherein said home doctor center gives each subscriber an advice in addition to a result of said initial diagnosis.

16. The home doctor system according to claim 1, 2 or 3 wherein said home doctor center supplies each subscriber with additional information based on a result of said initial diagnosis.

17. The home doctor system according to claim 16 wherein said additional information is helpful for prevention and treatment of diseases.

18. The home doctor system according to claim 9 wherein risks of payments of insured amounts to individual subscribers are calculated, and premiums are determined based on the calculated risks.

19. The home doctor system according to claim 9 wherein risks of payments of insured amounts to individual subscribers are calculated, and premiums are determined based on the calculated risks, and premiums are modified in response to changes of said calculated risks.

20. A blood capsule comprising:
a plurality of capsules connected by hollow pipes; and
a seal portion for sealing the interior of said hollow pipes and said capsules in a reduced pressure condition,
wherein a needle of a blood-collecting injection appliance penetrates said seal portion and introduces blood into said plurality of capsules, and
wherein said capsules are cut and separated into discrete capsules and set in a blood test apparatus.

21. An injection appliance for injection or collection of blood comprising:
an enlarged opening at a tip end of a needle.

22. An injection appliance for injection or collection of blood comprising:
a stopper located at a predetermined distance from a tip end of a needle.

23. An injection appliance for injection or collection of blood comprising:
a plurality of needles.

24. An injection appliance for injection or collection of blood comprising:
a needle unit and a main body or a capsule connected by a flexible tube.

25. The injection appliance according to claim 24 wherein said needle unit and said main body are detachably connected.
